SEQUENCE LISTING (2 sequences)

SEQ ID NO: 1 - DNA, 4374 bp, Artificial
  B domain deleted FVIII
atgcaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggttcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagacgcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatccgcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttacgaagatatttcagcatacttgctgagtaaaaacaatgccattgaacctaggagcttctctcagaatccaccagtcttgaaacgccatcaacgggaaataactcgtacgactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctactga

SEQ ID NO: 2 - protein, 1457 aa, Artificial
  Synthetic Construct
MQIELSTCFFLCLLRFCFSATRRYYLGAVELSWDYMQSDLGELPVDARFPPRVPKSFPFNTSVVYKKTLFVEFTVHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

(19) United States
(12) Patent Application Publication
(10) Pub. No.: US 2016/0220644 A1
(43) Pub. Date: Aug. 4, 2016

(54) METHOD OF TREATING HAEMOPHILIA BY INDUCING TOLERANCE TO BLOOD FACTORS

(71) Applicants: DBV TECHNOLOGIES, BAGNEUX (FR); INSERM (INSTITUT NATIONAL DE LA SANTE ET DE LA RECHERCHE MEDICALE), PARIS (FR)

(72) Inventors: SÉBASTIEN LACROIX-DESMAZES, PARIS (FR); LUCIE MONDOULET, CHATILLON (FR); VINCENT DIOSZEGHY, VILLIERS SUR MARNE (FR)

(21) Appl. No.: 15/135,914

(22) Filed: Apr. 22, 2016

Related U.S. Application Data
(63) Continuation-in-part of application No. PCT/EP2014/072508, filed on Oct. 21, 2014.

(30) Foreign Application Priority Data
Oct. 22, 2013 (EP) 13306453.5

(51) Int. Cl. A61K 38/37; A61K 9/70; A61K 9/00

(52) U.S. Cl. CPC A61K 38/37; A61K 9/0014; A61K 9/7023

(57) ABSTRACT

The present invention relates to the treatment of haemophilia. More specifically, the invention relates to a new method of inducing an immune tolerance against at least one blood factor used to treat haemophilia through the epicutaneous route by application of a skin patch device comprising a blood factor in order to continue haemophilia treatment. The present invention also relates to the skin patch device containing Factor VIII.

### CROSS-REFERENCE TO RELATED APPLICATION

This application is a continuation-in-part of International Patent Application No. PCT/EP2014/072508, filed Oct. 21, 2014.

The Sequence Listing for this application is labeled “Seq-List.txt” which was created on Apr. 21, 2016 and is 34 KB. The entire content of the sequence listing is incorporated herein by reference in its entirety.

### FIELD OF THE INVENTION

The present invention relates to a method of treating Haemophilia by inducing tolerance to blood factors. More specifically, the invention relates to a new method of treating haemophilia through the epicutaneous route.

In particular, the method of the invention relates to a method of inducting tolerance to factor VIII in a subject having haemophilia A.

More particularly, the method of the invention comprises applying to an area of the skin of a subject in need thereof a skin patch device comprising factor VIII, under conditions allowing a contact between said composition and the skin. The present invention also relates to the skin patch device containing Factor VIII (also referred to as FVIII).

### BACKGROUND OF THE INVENTION

Haemophilia (hemophilia) is a genetic disorder characterized by spontaneous hemorrhage or prolonged bleeding due to factor VIII or IX deficiency.

Haemophilia A (HA) is a rare X chromosome-linked recessive hemorrhagic disorder that concerns one individual in 5000 to 10000. Genetic abnormalities in the gene encoding factor VIII result in the absence of production of FVIII or in the production of defective FVIII molecules. Two thirds of the cases of HA are inherited. The remaining third of the patients do not have a family history of HA. In the latter patients, the defect in the FVIII-encoding gene has arisen de novo on the X chromosome. Several severities of HA may be distinguished that reflect the residual activity of FVIII measurable in the plasma, and are directly linked to the type of haemophilia-causing mutation.

Thus, patients with severe HA present with undetectable FVIII activity (<1%), while patients with moderate or mild HA have 1 to 5%, or 5 to 35% of normal values, respectively. While life-threatening bleedings are rare in patients with mild or moderate HA, severe HA is a crippling hemorrhagic disease, with elevated morbidity and mortality. To date, and despite the recent progress in gene therapy, no treatment is available to cure HA.

Hence, the treatment or prevention of bleeding episodes in the patients is achieved by replacement therapy using exogenous FVIII. Treatment revolves around substitution therapy with plasma derivatives or genetically engineered recombinant alternatives. Treatment may be administered after a hemorrhage (treatment on demand) or to prevent bleeding (prophylactic treatment).

Several forms of factor VIII have been used or are intended to be used as active substance for treating haemophilia. These include human plasma-based factor VIII like the active principles of Humate® P, Monoclate® P, Irnmunate® or Hemofil® M; recombinant human factor VIII, like the B-domainless Factor VIII (moroctocog alfa) which is described in PCT patent application WO 91/09122 and is the active principle ReFacto®. Other recombinant human factor VIII include the active principles of Kogenate® or Recombinate®, (octogog alpha) or porcine factor VIII (which was the active principle of the product Hyate.C® sold by Ipsen, Inc., USA) or recombinant full-length or truncated porcine factor VIII like the modified B-domainless form of porcine factor VIII disclosed in patent application WO 01/68109.

Modified Factor VIII proteins like the demannosylated recombinant Factor VIII disclosed in WO 2008/129422 can also be used for treating haemophilia.

Fragments like the peptides comprising between 8 and 15 amino acids disclosed in WO2006/003183 or the peptides disclosed in WO 2009/095646 can also be used as active substance for treating haemophilia. Domains A2 and/or C2 of factor VIII can also be used in the method of the invention.

In up to 30% of the patients however, replacement therapy is complicated by the occurrence of anti-drug antibodies, referred to as inhibitory anti-FVIII antibodies (or FVIII inhibitors), that preclude the use of FVIII. Inhibitory anti-FVIII antibodies are of the IgG isotype, and mostly of IgG1 and IgG4 subclasses. Anti-FVIII IgG are polyclonal in each patient. Several mechanisms have been described by which anti-FVIII IgG reduce the efficacy of therapeutically administered FVIII: inhibitory anti-FVIII IgG are directed to functional epitopes of FVIII and prevent, by steric hindrance, its interaction with different molecules participating in the coagulation cascade (e.g., von Willebrand factor, activated factor IX, factor X or phospholipids); non-inhibitory anti-FVIII IgG may form immune complexes with the therapeutically administered FVIII, thus accelerating its removal from the circulation and decreasing its half-life.

The presence in the blood of patients with haemophilia A of CD4+ T lymphocytes that proliferate when stimulated in vitro with FVIII or FVIII-derived peptides has been documented. Furthermore, analyses in an experimental model of severe haemophilia A have demonstrated that abrogation of the T-cell help using antibodies to the ligand for CD40 (CD40L or CD154) or using CTLA4-Ig constructs prevent the anti-FVIII immune response. Similarly, administration of anti-CD40L antibodies was found to block anamnestic responses to FVIII in some patients with haemophilia A, although the clinical trial was interrupted due to thrombotic complications fatal to the patients.

Correcting hemostasis in bleeding inhibitor-positive patients requires the use of bypassing agents such as activated prothrombin concentrates or activated recombinant factor VII. The occurrence of an inhibitor to therapeutic FVIII in a patient increases treatment costs by more than 3-fold, reaching 200,000 [symbol] /year in developed countries.

Immune tolerance induction (ITI) is the only available treatment that allows efficient eradication of FVIII inhibitors in patients with haemophilia A. ITI consists in the repeated administration of high dose therapeutic FVIII. It is successful in 60 to 80% of the patients. However, ITI is complicated by the facts that it requires an extreme compliance of the patients and that the cost of treatment may reach more than 0.2 million euros/patient/year. Several parameters have been associated with ITI outcome or with the duration of treatment until ITI success. These parameters include the age of the patients at start of ITI, the high or low dose FVIII treatment, the recombinant or plasmatic origin of the FVIII used for ITI, and the properties of the FVIII inhibitor.

Several approaches have been investigated using a mouse model of severe haemophilia A to induce tolerance to exogenous FVIII. Lei et al. (Blood. 2005;105: 4865-4870) demonstrated that FVIII-specific tolerance may be induced in FVIII-deficient mice by lipopolysaccharide (LPS)-activated B-cell blasts transduced with immunoglobulin (IgG)-FVIII fusion constructs. In an alternative strategy, the adoptive transfer of autologous apoptotic fibroblasts transfected with FVIII encoding gene was shown to induce FVIII-specific tolerance. More recently, Moghimi et al. (J Thromb Haemost 2011; 9: 1524-33) have shown that prophylactic immune tolerance protocol for FVIII can be developed using rapamycin.

Despite these preliminary results, cellular therapies remain empirical, hard to use in large scale on human, very expensive and the use of immunosuppressive drugs may expose the patients to opportunistic diseases.

Consequently, there is a real need for compositions and methods of treating haemophilia which are effective, safer and more patient friendly.

### SUMMARY OF THE INVENTION

The present invention provides a new method of treating and/or preventing haemophilia and/or the symptoms of haemophilia. More specifically, the invention shows, for the first time, that efficient treatment of haemophilia can be achieved through epicutaneous immunotherapy.

In particular, the method of the invention comprises applying to an area of the skin of a subject in need thereof a skin patch device comprising a blood factor, preferably Factor VIII, under conditions allowing a contact between said substance and the skin. The present invention shows that such an application provokes a tolerance to exogenous (therapeutic) factor VIII, leading to a very substantial decrease of the symptoms of haemophilia and an improved treatment efficacy.

An object of this invention thus resides in a method of treating and/or preventing haemophilia and/or the symptoms of haemophilia in a subject, said method comprising applying to an area of the skin of the subject a skin patch device comprising a blood factor, preferably Factor VIII (hereinafter also referred to as the “active substance”) under conditions allowing a contact between said active substance and the skin.

A further object of this invention relates to an occlusive skin patch device comprising a blood factor, preferably Factor VIII, in dry form, adhering to the patch through electrostatic forces, and its use in the treatment and/or prevention of haemophilia and/or the symptoms of haemophilia in a subject.

The invention also relates to the use of a skin patch device comprising a blood factor, preferably Factor VIII, in dry form, adhering to the patch through electrostatic forces, in the manufacture of a composition for treating and/or preventing haemophilia and/or the symptoms of haemophilia.

A further object of this invention is a method of treating and/or preventing haemophilia in a subject, the method comprising applying a blood factor, preferably Factor VIII, in dry form, to one or several areas of the skin of the subject under conditions allowing a contact between said blood factor and the skin and maintaining the blood factor in contact with the skin under conditions sufficient to cause a decrease of the immunoreactivity of the subject.

A further object of this invention is a method of decreasing the immunoreactivity of a subject having haemophilia, the method comprising applying a blood factor, preferably Factor VIII, in dry form, to one or several areas of the skin of the subject and maintaining the blood factor in contact with the skin under conditions sufficient to cause a decrease of the immunoreactivity of the subject.

A further object of this invention is a method of treating haemophilia in a subject in need thereof, the method comprising (i) administering exogenous factor VIII to the subject and (ii) epicutaneously applying factor VIII to one or several areas of the skin of the subject under conditions sufficient to induce tolerance to, or to reduce neutralization of exogenous factor VIII.

A further object of this invention is an improved method of treating haemophilia in a subject receiving exogenous Factor VIII, the improvement residing in epicutaneously applying factor VIII to one or several areas of the skin of the subject in order to induce tolerance to or to reduce neutralization of exogenous factor VIII.

A further object of this invention is a method of treating and/or preventing haemophilia in a subject, the method comprising applying a blood factor, preferably Factor VIII, in dry form, to one or several areas of the skin of the subject.

A further object of this invention relates to a composition comprising a blood factor, preferably Factor VIII, as well as to the use thereof for treating or preventing haemophilia in a subject in need thereof by epicutaneous application.

A further object of this invention relates to a composition comprising a blood factor, preferably Factor VIII for use for reducing bleeding in a subject in need thereof by epicutaneous application.

### LEGEND TO THE FIGURES

FIG. 1: Study design in KO-hemophilia mice to evaluate the induction of tolerance to FVIII by delivering epicutaneously exogenous FVIII: empty (placebo control group), plasma-derived FVIII (pdFVIII), recombinant B domain deleted FVIII (FVIII-HSQ) and recombinant B-domain deleted FVIII combined with Aryl hydrocarbon receptor (AhR) ligand. The treatment has consisted to a 48 h-Viaskin patch application weekly for 8 weeks. Blood samples collected at the end of treatment and after 4 injections of FVIII were evaluated for the capacity to inhibit FVIII.

FIG. 2: Measurement of the inhibitory activity by Bethesda assay of plasma sampled from KO-hemophilia mice after 4 injections of FVIII. Results are expressed in BU/mL. Statistical differences are based on a Mann-Whitney test.

### DETAILED DESCRIPTION OF THE INVENTION

The present invention relates to a method for treating and/or preventing haemophilia and/or the symptoms of haemophilia in a subject using epicutaneous administration.

Haemophilia is a genetic disorder characterized by spontaneous hemorrhage or prolonged bleeding due to factor VIII (haemophilia A) or IX (haemophilia B) deficiency. Annual incidence is estimated at 1/5,000 male births and the prevalence is estimated at 1/12,000 in France. The severity of the clinical manifestations depends on the extent of the coagulation factor deficiency. Diagnosis is made on the basis of coagulation tests revealing prolonged blood coagulation times. The type and severity of the haemophilia are determined through specific measurements of factor VIII levels.

When used in the present application, the terms “Factor VIII” or “FVIII” mean full length or truncated, plasma-based or recombinant factor VIII, or recombinant domains of factor VIII and in particular one of the above mentioned products or a combination thereof. The terms “Factor VIII” or “FVIII” thus include any polypeptide having an activity of factor VIII. The preferred form of Factor VIII to be used in the method of the invention is the B-domainless Factor VIII (moroctocog alfa) which is also referred to as B domain-deleted FVIII (FVIII-HSQ) below. The amino acid sequence of a preferred example of a B domain-deleted VIII is presented as SEQ ID NO: 2. Any polypeptide having at least 90% sequence identity to SEQ ID NO:2, preferably at least 95%, 96%, 97%, 98%, or more, may be used as a particular embodiment of the invention. Other particular forms of Factor VIII that may be used in the method of the invention include domains A2 and/or C2 of factor VIII.

The term “exogenous factor VIII” designates any therapeutic factor VIII used to cure a subject, typically by injection.

In particular, the method of the invention relates to a method of treating and/or preventing haemophilia A and/or the symptoms of haemophilia A in a subject, said method comprising applying to at least one area of the skin of the subject a skin patch device comprising a composition comprising an active substance that causes a thrombin production, preferably a blood factor, most preferably factor VIII, under conditions allowing a contact between said composition and the skin.

When suffering from factor VIII deficiency, patients generally receive exogenous (therapeutic) FVIII injections as treatment. Unfortunately, several years after the beginning of the treatment, an immune response to said exogenous (i.e., non-self or “foreign”) factor VIII appears, notably by the production of anti-factor VIII antibodies, leading to the destruction or neutralization of the exogenous factor VIII. The present invention allows to effectively lower said immune response to “exogenous” factor VIII, leading to a substantially improved or restored therapeutic efficacy of the exogenous factor VIII to cure the patient.

The invention thus relates to a blood factor, preferably factor VIII, for use in a method of treating haemophilia A by epicutaneous application to a subject in need thereof, particularly to a subject receiving exogenous factor VIII. The invention also relates to a skin patch device comprising a blood factor, preferably factor VIII, as well as its use in a method of treating haemophilia A in a subject in need thereof.

In a particular embodiment, the method of the invention comprises the induction of an immune tolerance against factor VIII, especially exogenous factor VIII, in the subject in need thereof.

In particular, the invention relates to a skin patch or method for the prevention or treatment of any adverse immune reaction directed to factor VIII, especially exogenous factor VIII, in a subject in need thereof. By reducing such adverse immune response, the invention restores and/or substantially increases the therapeutic efficacy of exogenous factor VIII in the subject.

In this regard, in a particular embodiment, the method of the invention relates to (i) a skin patch device comprising at least one blood factor, preferably factor VIII, in combination with (ii) an injectable factor VIII composition, for use to treat haemophilia in a subject in need thereof by simultaneous or sequential administration. In a particular embodiment, the method comprises applying a skin patch device comprising at least one blood factor, preferably factor VIII, to the subject in need thereof, and subsequently injecting at least one blood factor, preferably factor VIII, to the subject. Alternatively, the skin patch device may be applied once an adverse immune response appears in the subject, to neutralize the same. In practice, both components of the method of the invention should be used as of the beginning of the treatment regimen, when possible.

In particular, the method of the invention relates to a skin patch device for its use as defined above which comprises repeated application of the device to at least one area of the skin of the subject under conditions allowing contact between the at least one blood factor, preferably factor VIII, and the skin and penetration of the at least one blood factor, preferably factor VIII into the epidermis.

In particular, the invention relates to a method or skin patch device for its use as defined above wherein the blood factor is factor VIII and is selected from the group consisting of full length or truncated factor VIII.

In particular, the invention relates to a method or skin patch device for its use as defined above wherein the blood factor is a recombinant factor VIII, preferably a recombinant B-domainless factor VIII.

In particular, the invention relates to a method or skin patch device for its use as defined above wherein the blood factor is a plasma-based factor VIII.

In a particular embodiment of the invention, the blood factor is in dry form. In a further particular embodiment, the blood factor is applied in absence of an adjuvant.

In particular, the invention relates to a skin patch device comprising at least one blood factor, preferably factor VIII, and possibly one or several carriers (e.g., excipients, diluents and the like).

In particular, the invention relates to a drug delivery system comprising:
a skin patch,
a blood factor, preferably factor VIII, and
possibly one or several carriers
wherein the skin patch is configured to be attached to a skin portion of an individual, and to needlelessly deliver the blood factor, preferably factor VIII, dermally to the individual.

The invention also relates to a kit comprising:
a skin patch comprising a blood factor, preferably factor VIII, and
an injectable composition comprising a blood factor, preferably factor VIII.

The invention has concerns a method of treating Haemophilia in a subject in need thereof comprising a repeated application of a skin patch device comprising at least one blood factor, preferably factor VIII, and possibly a pharmaceutically acceptable carrier, to at least one area of the skin of the subject under conditions allowing contact between the at least one blood factor, preferably factor VIII and the skin and penetration of the at least one blood factor, preferably factor VIII into the epidermis.

In a specific aspect of the invention, the application of said skin patch device comprising the blood factor to the skin causes a decrease of the immunoreactivity of the subject. By “a decrease of the immunoreactivity” is meant that the immune response to exogenously injected blood factor, preferably factor VIII is lowered. The patch may be used prior to treatment with exogenous blood factor, or at any time during the course of said treatment. The method of the invention is based on an immunotherapy process occurring by the application of exogenous factor blood factor, preferably VIII to the skin of the patient suffering of haemophilia A.

The invention advantageously shows that such a method causes a substantial decrease of immunoreactivity and potentializes exogenous factor VIII activity.

The invention may be used to treat any type of haemophilia A including, without limitation, mild haemophilia A, moderately severe haemophilia A and severe haemophilia A. In a preferred embodiment of the invention, said haemophilia A is severe haemophilia A.

Haemophilia A is the most common form of haemophilia characterized by spontaneous or prolonged hemorrhages due to factor VIII deficiency. Transmission is X-linked recessive and the disorder is caused by mutations in the F8 gene (Xq28) encoding coagulation factor VIII. The severity of the clinical manifestations depends on the extent of the factor VIII deficiency.

Mild haemophilia A is a form of haemophilia A characterized by a small deficiency of factor VIII leading to abnormal bleeding as a result of minor injuries, or following surgery or tooth extraction. Mild haemophilia A accounts for around 40% of all cases of haemophilia A. The biological activity of factor VIII is between 5 and 40%. Spontaneous hemorrhages do not occur.

Moderately severe haemophilia A is a form of haemophilia A characterized by factor VIII deficiency leading to abnormal bleeding as a result of minor injuries, or following surgery or tooth extraction. Moderately severe haemophilia A accounts for around 20% of all cases of haemophilia A. The biological activity of factor VIII is between 1% and 5%. Spontaneous hemorrhages are rare.

Severe haemophilia A is a form of haemophilia A characterized by a large deficiency of factor VIII leading to frequent spontaneous hemorrhage and abnormal bleeding as a result of minor injuries, or following surgery or tooth extraction. Severe haemophilia A accounts for around 40% of all cases of haemophilia A. The biological activity of factor VIII is below 1%. Severe haemophilia A is generally fatal during childhood or adolescence.

With the invention, the activity of exogenous factor VIII may be restored or increased by at least 10%, 20%, 30%, 40%, 50%, or more as compared to the residual activity in the subject.

In the context of severe HA, where the activity of factor VIII is generally below 1%, the method of the invention can restore such an activity to a substantial level above 10%, even above 30%, or more remarkably above 50%. Any such level is suitable to avoid spontaneous hemorrhages and to reduce bleeding. Above 50% is sufficient to restore normal bleeding in the subject and avoid any complication of hemophilia.

In a particular embodiment, the method comprises a repeated application of a skin patch device, typically over prolonged periods of time. For instance, the method typically involves the sequential application of at least 2 patches over a period of 1 month or more. Application may be repeated until tolerance is established. It may then be stopped, or interrupted, or spaced, according to the patient tolerance.

The invention relates to a skin patch device comprising a blood factor, preferably factor VIII, for use in a method of treating haemophilia in a subject in need thereof, by repeated application of the device to at least one area of the skin of the subject under conditions allowing contact between the blood factor, preferably factor VIII and the skin and penetration of the blood factor, preferably factor VIII into the epidermis.

A particular object of this invention relates to an occlusive skin patch device comprising Factor VIII in dry form, for treating haemophilia A. In a further particular embodiment, Factor VIII is maintained on the patch through electrostatic forces, wherein said patch is applied to at least one area of the skin of the subject under conditions allowing a contact between said composition and the skin.

A further embodiment of the present invention resides in a use of an occlusive patch device described above, in the manufacture of a composition for treating haemophilia A.

The present invention provides a new epicutaneous immunotherapy method for treating haemophilia A, which comprises repeatedly administering to said subject a composition via the epicutaneous route by means of a skin patch device comprising Factor VIII as the active substance and a backing, the periphery of said backing being adapted to create with the skin a hermetically closed chamber, wherein the backing bears on its skin facing side within the chamber said one or more proteins in a dose sufficient to decrease the skin reactivity in said subject following application of the patch device to the skin, said composition being removed from the backing following application of the patch device to the skin and thereafter delivered to the subject via the epicutaneous route, said administration leading, on repetition, to a progressive decrease against immunoreactivity to exogenous factor VIII.

In another aspect, the present invention also concerns the use of a skin patch device comprising a blood factor, preferably factor VIII as the active substance and a backing, the periphery of said backing being adapted to create with the skin a hermetically closed chamber, wherein the backing bears on its skin facing side within the chamber a blood factor, preferably factor VIII in the manufacture of a composition for treating haemophilia in a subject. The invention may be used in any subject, for example animal or human subject, and particularly any human subject, including children and adults. Preferably, the subject suffers from haemophilia A.

The immunotherapeutic method of the invention involves the administration of a composition containing a blood factor, preferably factor VIII to a subject via the epicutaneous route using particular patch devices, leading to decreasing the immunoreactivity.

As used in this specification, the term “epicutaneous route” means the administration of an active substance to a subject by application of this active substance on the skin. The epicutaneous route does not require the use of a needle, syringe or of any other means to perforate or to alter the integrity of the superficial layer of the epidermis. The active substance is maintained in contact with the skin for period of time and under conditions sufficient to allow the active substance to penetrate into the stratum corneum of the epidermis.

The term “treating” includes any reduction, decrease, or attenuation of the severity of haemophilia A. For instance, treating includes transforming a severe form of haemophilia A in a subject to a mild or moderately severe form.

The term “treating” also includes a reduction of the symptoms of haemophilia A, and notably to the bleeding, not only in intensity but also in duration. The symptoms of haemophilia A are notably frequent spontaneous hemorrhage and abnormal bleeding as a result of minor injuries, or following surgery or tooth extraction. Bleeding most often occurs around the joints (hemarthroses) and in the muscles (hematomas). Spontaneous hematuria is a fairly frequent and highly characteristic sign of the disorder. Treating thus includes a reduction in bleeding time in a subject, and/or a reduction in bleeding intensity in a subject.

By “a reduction of the immunoreactivity” is meant that the immune response to the exogenously injected blood factor, preferably factor VIII is lowered.

In a preferred embodiment, the factor VIII used in the invention is selected from full length factor VIII protein, truncated factor VIII (wherein part of the protein is missing) or domains of factor VIII such as A2 and C2 domains, or any mixture thereof. In a particular embodiment, Factor VIII or truncated FVIII are of human origin. In a preferred embodiment, Factor VIII is the recombinant B-domainless factor VIII. The factor VIII may be used in native form, or modified e.g., chemically, enzymatically and/or thermally.

The blood factor composition may be in liquid form, if appropriately protected against the action of proteases, such as a solution or a dispersion of particles. In that case, effective epicutaneous administration is ensured by migration of the active substance from the liquid phase of the composition to the skin in order to allow the active substance to penetrate into the stratum corneum of the epidermis. In a particular embodiment, the migration of the active substance from the liquid phase of the composition is ensured by diffusion of the active substance through the condensation formed within the hermetically closed chamber, e.g. as a result of perspiration.

In a preferred embodiment, the blood factor composition is in dry form, in particular in a particulate form, obtainable, for example, by lyophilisation. The use of proteins in dry form is advantageous. Indeed, such particulate active substance may be directly attached to the backing of the device, thereby avoiding any chemical interaction or any reaction which might disturb the immunogenicity of these proteins. Moreover, the use of the particles allows preserving the substance in a suitable packaging, such that there is no longer any need to carry out an extemporaneous preparation. In this case, the epicutaneous administration of active substances held on the backing of the patch may be ensured by dissolution of these active substances in the condensation formed within the hermetically closed chamber.

The active substance composition may further comprise additional components, such as pharmaceutical acceptable excipients or carriers as those disclosed in Handbook of Pharmaceutical Excipients, sixth edition 2009, Rowe et al., Pharmaceutical Press. Suitable excipients or carriers are well known to those of skill in the art and include, without limitation, sterile saline, lactose, sucrose, calcium phosphate, gelatin, dextrin, agar, pectin, peanut oil, olive oil, sesame oil and water. Additionally, the carrier or diluent may include a time delay material, such as glyceryl monostearate or glyceryl distearate alone or with a wax.

In an embodiment, the composition used in the present invention is formulated without any adjuvant.

In another embodiment, the active substance composition used in the present invention may comprise or may be applied with an adjuvant. Within the context of this invention, an adjuvant designates any substance that acts to activate, accelerate, prolong, or enhance active substance-specific immune responses when used in combination with a specific active substance.

The skin patch device used in the method of the invention preferably comprises a backing, the periphery of said backing being adapted to create with the skin a hermetically closed chamber. This backing bears on its skin facing side within the chamber the composition used to decrease the immunoreactivity.

Preferably, the periphery of the backing has adhesive properties and forms an airtight joint to create with the skin a hermetically closed chamber.

In a particular embodiment, the active substance(s) is (are) maintained on the backing by means of electrostatic and/or Van der Waals forces. This embodiment is particularly suited where the active substances present in the composition are in solid or dry form (e.g., particles), although it may also be used, indirectly, where the blood factor, preferably factor VIII is in a liquid form.

Within the context of the present invention, the term “electrostatic force” generally designates any non-covalent force involving electric charges. The term “Van der Waals forces” designates non-covalent forces created between the surface of the backing and the solid allergen, and may be of three kinds: permanent dipoles forces, induced dipoles forces, and London- Van der Waals forces. Electrostatic forces and Van der Waals forces may act separately or together.

In this respect, in a preferred embodiment, the patch device comprises an electrostatic backing. As used herein, the expression “electrostatic backing” denotes any backing made of a material capable of accumulating electrostatic charges and/or generating Van der Waals forces, for example, by rubbing, heating or ionization, and of conserving such charges. The electrostatic backing typically includes a surface with space charges, which may be dispersed uniformly or not. The charges that appear on one side or the other of the surface of the backing may be positive or negative, depending on the material constituting said backing, and on the method used to create the charges. In all cases, the positive or negative charges distributed over the surface of the backing cause forces of attraction on conducting or non-conducting materials, thereby allowing to maintain the blood factor, preferably factor VIII. The particles also may be ionized, thereby causing the same type of electrostatic forces of attraction between the particles and the backing. Examples of materials suitable to provide electrostatic backings are glass or a polymer chosen from the group comprising cellulose plastics (CA, CP), polyethylene (PE), polyethylen terephtalate (PET), polyvinyl chlorides (PVCs), polypropylenes, polystyrenes, polycarbonates, polyacrylics, in particular poly(methyl methacrylate) (PMMA) and fluoropolymers (PTFE for example). The foregoing list is in no way limiting. The back of the backing may be covered with a label which may be peeled off just before application. This label makes it possible, for instance, to store the composition containing the blood factor, preferably factor VIII in the dark when the backing is at least partially translucent.

The intensity of the force between a surface and a particle can be enhanced or lowered by the presence of a thin water film due to the presence of moisture. Generally, the patch is made and kept in a dry place. The moisture shall be low enough to allow the active ingredient to be conserved. The moisture rate can be regulated in order to get the maximum adhesion forces. As discussed above, the use of an electrostatic backing is particularly advantageous where the blood factor, preferably factor VIII is in a dry form, e.g., in the form of particles. Furthermore, the particle size may be adjusted by the skilled person to improve the efficiency of electrostatic and/or Van der Waals forces, to maintain particles on the support.

In a specific embodiment, the patch comprises a polymeric or metal or metal coated polymeric backing and the particles of active substances present in the composition are maintained on the backing essentially by means of Van der Waals forces. Preferably, to maintain particles on the support by Van der Waals forces, the average size of the particles is lower than 60 micrometer. In another embodiment, the blood factor, preferably factor VIII is maintained on the backing by means of an adhesive coating on the backing. The backing can be completely covered with adhesive material or only in part. Different occlusive backings can be used such as polyethylene or PET films coated with aluminium, or PE, PVC, or PET foams with an adhesive layer (acrylic, silicone, etc.).

Preferred examples of patch devices for use in the present invention are disclosed in WO 02/071950 or U.S. Pat. No. 7,635,488 (Viaskin®).

Other examples are disclosed in WO 2009/095591, which also discloses a spray-drying process to load the substance in particulate form on the backing of a patch device. An electrospray device uses high voltage to disperse a liquid in the fine aerosol. Proteins dissolved in a solvent are then pulverized on the patch backing where the solvent evaporates, leaving allergens in particles form. The solvent may be, for instance, water or ethanol, according to the desired evaporation time. Other solvents may be chosen by the skilled person. This type of process to apply substances on patch backing allows nano-sized and mono-sized particles with a regular and uniform repartition of particles on the backing. This technique is adapted to any type of patch such as patch with backing comprising insulating polymer, doped polymer or polymer recovered with conductive layer. Preferably, the backing comprises a conductive material.

In another embodiment, the periphery of the backing is covered with a dry hydrophilic polymer, capable of forming an adhesive hydrogel film by contact with the moistured skin (as described in WO2009/050403). In this embodiment, the skin has to be moistured before the application of the patch. When the hydrogel comes into contact with the moistured skin, the polymer particles absorb the liquid and become adhesive, thereby creating a hermetically closed chamber when the patch is applied on the skin. Examples of such hydrogels include polyvinylpyrolidone, polyacrylate of Na, copolymer ether methyl vinyl and maleic anhydride.

In another particular embodiment, the liquid composition comprising the active substances is held on the support of the patch in a reservoir of absorbent material. The composition may consist in an allergen solution or in a dispersion of the allergens, for example in glycerine. The adsorbent material can be made, for example, of cellulose acetate.

The backing may be rigid or flexible, may or may not be hydrophilic, and may or may not be translucent, depending on the constituent material. In the case of glass, the support may be made break-resistant by bonding a sheet of plastic to the glass.

In one embodiment, the backing of the patch contains a transparent zone allowing directly observing and controlling the inflammatory reaction, without necessarily having to remove the patch. Suitable transparent materials include polyethylene film, polyester (polyethylene-terephtalate) film, polycarbonate and every transparent or translucent biocompatible film or material.

In a particular embodiment, the portion of the backing bearing the allergen is not in direct contact with the skin. In this embodiment, the height of the chamber defined by the backing, the periphery of the backing and the skin is in the range of 0.1 mm to 1 mm.

The skin patch device is preferably non-perforating, allowing penetration or contact of the blood factor through passive diffusion.

The method of the invention typically involves the repeated application of a device according to the invention to the subject as disclosed above, leading to a progressive decrease of the immunoreactivity in the subject. The specific dose of the active substance as well as the number of applications and duration of contact can be adapted by the skilled artisan, depending on the severity of haemophilia A, the subject, the nature of the blood factor preparation, the type of patch device used, etc.

Generally, the method comprises the application of patch devices as disclosed above preferably at least 3, 5, 10, 15 times, or more, over a period of time which may be comprised between a week and months or years. The treatment may be stopped at any time, e.g., once severe haemophilia A has been reduced to mild or moderately severe forms, or once bleeding time or intensity has been sufficiently reduced, or e.g., more generally once the practitioner determines that treatment can be stopped.

The amount of active substance on each patch is typically in the range of 0.1 to 1000 μg/cm of patch surface, preferably in the range of 20 to 500 μg/cm of patch surface, more preferably in the range of 20 to 200 μg/cm² of patch surface. The patch surface is in the range of 1 cm to 10 cm, preferably in the range of 1 cm to 5 cm.

For application, the patch devices may be applied directly to the skin, without any pre-treatment, preferably on a hairless part of the body. Alternatively, the skin may be treated prior to application of the device, to disrupt the stratum corneum, to remove hairs or simply to cause hydration of the skin, at the site of contact with the patch device. As disclosed in the experimental section, the method of the invention results in a progressive decrease of the skin reactivity of the subject.

Patient having haemophilia A may be treated by the epicutaneous application of several skin patch devices, each containing a specific active substance composition, and/or by the application of a device comprising a combination of these active substances. In this regard, in a particular embodiment, for treating haemophilia A in a patient in need thereof, 2 devices may be applied by the epicutaneous route, one containing a full length FVIII allergen composition and the other containing a truncated FVIII composition. When several patches comprising distinct active substances are used, they may be applied simultaneously or sequentially, or both. Typically, they are applied under conditions allowing a contact with the skin during a common period of time.

The present invention also relates to the use of a skin patch device as described above, in the manufacture of a composition for treating haemophilia A in a subject. The invention may be used in any mammalian subject, particularly in human subjects.

The following examples are given for purposes of illustration and not by way of limitation.

### EXAMPLES

### 1—Preparation and Purification of B Domain-deleted FVIII (FVIII-HSQ)

A two-step ion-exchange chromatography procedure is used to isolate HSQ from conditioned serum-free medium. Briefly, HSQ-containing medium is loaded onto a HiLoad 26/10 sp Sepharose HP equilibrated in 0.15 MNaCl, 20 mM HEPES, 5 mM CaCl₂, 0.01% Tween 80, pH 7.4. HSQ is eluted with a linear 0.2-0.65 M NaCl gradient in the same buffer. Fractions containing FVIII are pooled, diluted to 0.15M NaCl in the same buffer, applied to a Resource Q protein liquid chromatography column, and eluted with a linear 0.2-1.0M NaCl gradient. Fractions analyzed by one-stage coagulation assay using Sysmex CA-500 Automated Coagulometer and absorbance at A ₂₈₀ and 4-12% SDS polyacrylamide gel electrophoresis. Fractions containing peak FVIII activity are pooled, buffer exchanged using Amican Ultracel centrifugal filters (10 kDa cutoff) against 20 mM Tris, 5 mM CaCl₂, 0.01% Tween 80, pH 7.4, and filtered using 0.2 μm syringe filter. Specific activity is calculated using a molar extinction coefficient determined from the absorbance at 280 nm and the predicted tyrosine, tryptophan, and cysteine content.

### 2—Preparation of Patch Devices Containing B Domain-deleted FVIII (FVIII-HSQ) or Plasma-derived FVIII (pd-FVIII) or FVIII+AhR (Aryl-hydrocarbone Receptors) Ligand

The patches used in the experiments reported below are manufactured according to the process disclosed in U.S. patent application No. US2010/0297213.

FVIII-HSQ (SEQ ID NO: 2) was eluted into a buffer containing 20 mM Tris, 5 mM Cacl2, 1 M NaCl and 0.01% Tween 80. A droplet (100 μL) containing 25 μg of FVIII-HSQ was loaded onto the Viaskin® patch by using a micropipette.

FVIII-HSQ in its initial buffer was mixed with AhR ligands (e.g. kynerunine) and a droplet of the mix was loaded onto the Viaskin® patch by using a micropipette.

Plasma-derived FVIII (pd-FVIII) was used without any specific preparation. A droplet containing 25 pg of FVIII was loaded onto the Viaskin® patch by using a micropipette.

The elution buffer of FVIII-HSQ, i.e., 20 mM Tris, 5 mM Cacl2, 1 M NaCl and 0.01% Tween 80, was loaded (50 nL) onto the Viaskin® patch by using a micropipette.

After the final deposition on all patches, these were dried at 30° C. for 2 h in a fan-assisted oven.

After drying period, all patches were covered with a protective film and primary packaging was performed as follows: five patches were put into a pouch of PET/Alu/PE film followed by the addition of N2 into the pouch and then thermo-sealing.

### 3—Efficacy in Vivo

The feasibility and efficacy of epicutaneous technique to treat haemophilia was evaluated in a FVIII-deficient mice model. Four groups of ten mice on the C57B1/6 background, aged 8 to 10-week old, are administered with 2 μg of human full length recombinant FVIII (FVIII-FL) intravenously. Subsequently, the mice are treated with a patch device as described in Example 2 containing 50 μg of B domain-deleted FVIII (FVIII-HSQ) (groups II), or 50 μg of plasma-derived FVIII (group III), or 50 μg of FVIII-HSQ+AhR ligands (group IV), or empty patches (group I) as negative controls. Treatment is performed for 8 consecutive weeks.

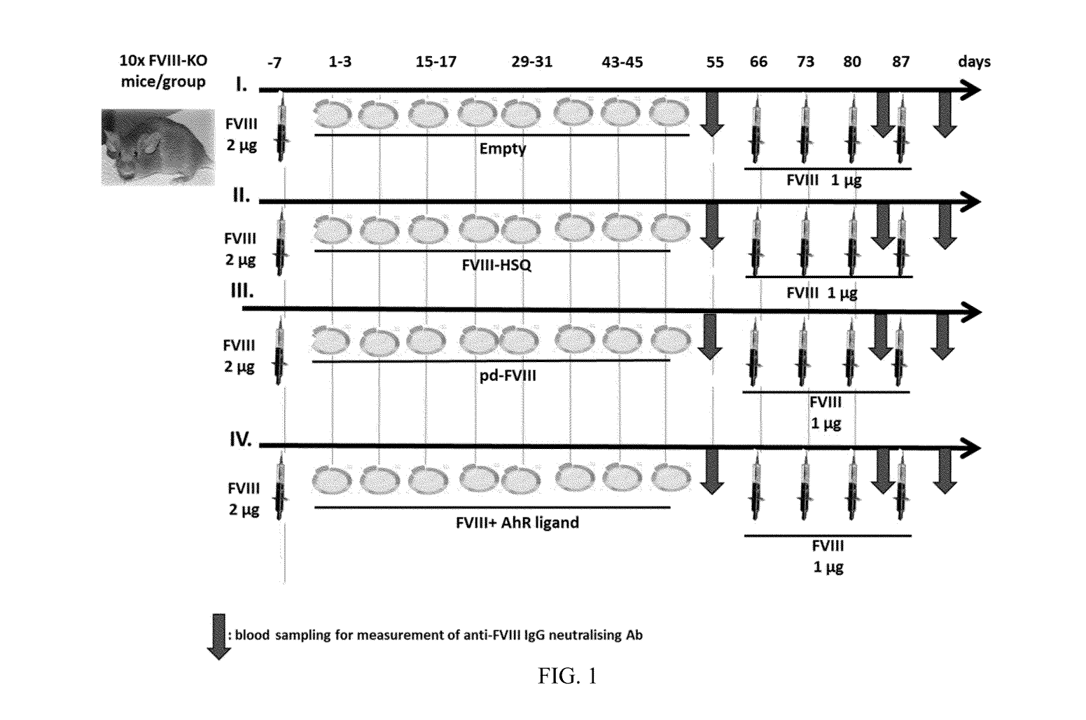

The four groups of mice are next treated once a week for four weeks with 1 μg FVIII-FL. Blood samples are harvested on days 55 before the four injections with 1 μg FVIII-FL, between the third and the fourth injections with 1 μg FVIII-FL and lastly on day 94, i.e., seven days after the fourth injection with 1 μg FVIII-FL. Details of the protocol are provided in FIG. 1.

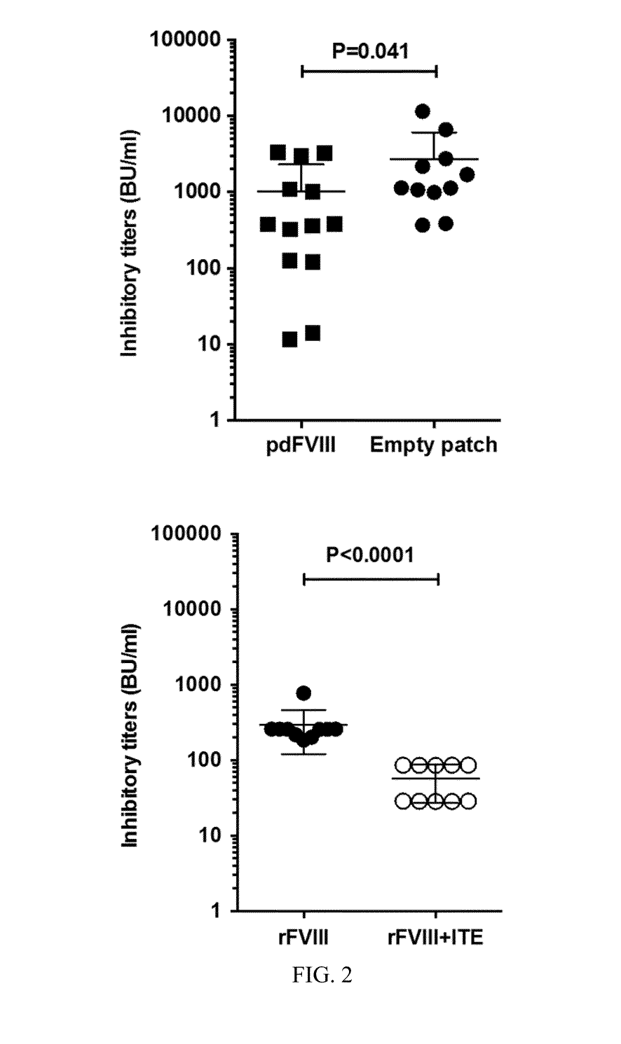

The results of these experiments allow to show that the epicutaneous application of factor VIII leads to a reduction of immunoreactivity in treated groups, which potentiates the effect of injected exogenous factor VIII, leading to an improved treatment of hemophilia A. In particular, the results of these experiments has confirmed the induction of tolerance to FVIII by delivering plasma-derived FVIII, recombinant B-domain deleted FVIII, or recombinant B-domain deleted FVIII combined with Aryl hydrocarbon receptor (AhR) ligand. The quantity of inhibitors was significantly decreased compared to empty patch treatment (FIG. 2). Recombinant B-domain deleted FVIII was particularly effective for epicutaneous induction of tolerance.

### 4—Evaluation of Humoral Anti-FVIII Immune Response

The humoral anti-FVIII immune response is assessed by ELISA. For detection of anti-FVIII IgG, ELISA plates (Nunc, Roskilde, Denmark) are coated overnight at 4° C. with recombinant FVIII (2 μg/ml, FVIII-HSQ). Plates are blocked with PBS and 1% BSA. Serum dilutions are then incubated for 1 h at 37° C. Bound IgG are revealed with a horseradish peroxidase-coupled monoclonal anti-mouse IgG (Southern Biotech) and substrate. The mouse monoclonal anti-FVIII IgG ESH8 (American Diagnostica, Stamford, Conn., USA) is used as a standard. Results are expressed either as micrograms per milliliter of anti-FVIII IgG ESH8 equivalent.

The results of this experiment allow to show that humoral anti-FVIII immune response is reduced in treated subjects.

### 5—Evaluation of FVIII Inhibitors

The level of FVIII inhibitors is assessed by a functional coagulation assay. Serum are incubated with standard human plasma (Dade- Behring, Marburg, Germany) for 2 h at 37° C. The residual procoagulant FVIII activity is measured with a chromogenic assay, following the manufacturers' recommendations (Dade-Behring). Bethesda titers, expressed in Bethesda units (BU) per milliliter of serum, are calculated as described previously. Bethesda titers are defined as the reciprocal of the dilution of serum that yields 50% residual FVIII activity.

The results of these experiments has confirmed the induction of tolerance to FVIII by delivering plasma-derived FVIII, recombinant B-domain deleted FVIII or recombinant B-domain deleted FVIII combined with Aryl hydrocarbon receptor (AhR) ligand.

1. A method of treating haemophilia A in a subject in need thereof, comprising applying to an area of skin of the subject a skin patch device comprising factor VIII.

2. The method of claim 1, wherein application of the device induces an immune tolerance against factor VIII in said subject.

3. The method of claim 1, further comprising simultaneously or sequentially injecting a blood factor VIII composition to the subject.

4. The method of claim 1, wherein the method comprises repeated application of the device to at least one area of the skin of the subject under conditions allowing contact between factor VIII and the skin and penetration of factor VIII into the epidermis.

5. The method of claim 1, for treating mild, moderately severe, or severe forms of haemophilia A.

6. The method of claim 1, wherein factor VIII is a full length or truncated factor VIII polypeptide.

7. The method of claim 6, wherein the factor VIII is a recombinant B-domainless factor VIII.

8. The method of claim 7, wherein the factor VIII comprises the amino acid sequence of SEQ ID NO: 2.

9. The method of claim 1, wherein the factor VIII is in dry form on the skin patch device.

10. The method of claim 1, wherein said factor VIII is applied in the absence of an adjuvant.

11. A skin patch device comprising Factor VIII and one or several pharmaceutically acceptable excipients or carriers.

12. A drug delivery system comprising:
a skin patch,
a blood factor, preferably Factor VIII, and
one or several carriers,
wherein the skin patch is configured to be attached to a skin portion of an individual, and to needlelessly deliver the blood factor, preferably factor VIII, dermally to a subject.

13. A kit comprising:
a skin patch comprising Factor VIII, and
an injectable composition comprising Factor VIII.
